(19) United States
(12) Patent Application Publication
(10) Pub. No.: US 2020/0121627 A1
(43) Pub. Date: Apr. 23, 2020

(54) COMPOSITION COMPRISING AZELAIC ACID OR AS ACTIVE INGREDIENTS FOR MUSCLE STRENGTHENING, DEVELOPMENT, DIFFERENTIATION, REGENERATION OR INHIBITING MUSCLE ATROPHY

(71) Applicant: INDUSTRY-ACADEMIC COOPERATION FOUNDATION, YONSEI UNIVERSITY, Seoul (KR)

(72) Inventor: Tae Sun PARK, Seoul (KR)

(73) Assignee: INDUSTRY-ACADEMIC COOPERATION FOUNDATION, YONSEI UNIVERSITY, Seoul (KR)

(21) Appl. No.: 16/654,805

(22) Filed: Oct. 16, 2019

(30) Foreign Application Priority Data
Oct. 18, 2018 (KR) 10-2018-0124733

(51) Int. Cl. A61K 31/20; A61K 8/362; A61K 8/31; A61K 8/92; A61K 8/86; A61K 8/34; A61K 8/41; A61P 21/00; A61Q 19/00

(52) U.S. Cl. CPC A61K 31/20; A61K 8/362; A61K 8/31; A61K 8/925; A61Q 19/00; A61K 8/345; A61K 8/41; A61P 21/00; A61K 8/86

(57) ABSTRACT

The present disclosure relates to a pharmaceutical composition for preventing or treating a muscle disease, comprising, as an active ingredient, azelaic acid or a pharmaceutically acceptable salt thereof. In muscle cells, the azelaic acid according to the present disclosure can increase expression of proteins associated with muscle protein synthesis and muscle mass increase and can inhibit expression of enzymes involved in muscle protein degradation; and therefore the azelaic acid can exhibit a muscle strength-enhancing effect through muscle differentiation, muscle regeneration, and muscle mass increase in a muscle disease caused by muscle function decline, muscle wasting, or muscle degeneration. In addition, the azelaic acid can inhibit muscle decrease. Accordingly, the azelaic acid can be used for preventing or treating a muscle disease, differentiating muscles, regenerating muscles and enhancing muscles, or for increasing muscle mass, promoting muscle production, or improving muscle function.

### CROSS-REFERENCE TO RELATED APPLICATIONS

This application claims priority to and the benefit of Korean Patent Application No. 10-2018-0124733, filed on Oct. 18, 2018, the disclosure of which is incorporated herein by reference in its entirety.

### FIELD OF THE DISCLOSURE

The present disclosure relates to a pharmaceutical composition for preventing or treating a muscle disease, comprising, as an active ingredient, azelaic acid or a pharmaceutically acceptable salt thereof.

### BACKGROUND

South Korea has entered an aging society as the elderly population accounted for 7.2% of the total population in 2000. In 2050, South Korea is expected to enter a super-aging society (elderly population being over 20%). Muscle mass in humans decreases with age (about 10 to 15% decrease at the ages of 50 to 70 years, and over 30% decrease at the ages of 70 to 80 years), and thus muscle strength and muscle function weaken, which is referred to as age-related sarcopenia. Age-related sarcopenia causes activity disorder and gait disorder, and thus becomes a major cause of limiting independent living of the elderly. In addition, sarcopenia decreases basal metabolic rate, which increases insulin resistance, promotes development of type 2 diabetes, and increases risk of developing hypertension and cardiovascular diseases by 3 to 5 times. No medicine is currently approved for use in the treatment of sarcopenia, and drug repositioning technology is being developed to apply, to sarcopenia, myostatin inhibitors or FDA-approved therapeutic agents for treating other diseases.

Muscles are divided into skeletal muscles, cardiac muscles, and visceral muscles. Among these, skeletal muscles are the most abundant tissues in the human body, accounting for 40 to 45% of body weight. Skeletal muscles are attached to bones through tendons, and play roles in creating bone movement or force. One muscle is made up of numerous myofibers which are in turn made up of numerous myofibrils composed of actin and myosin. When actin and myosin move in an overlapping manner to each other, muscle length is shortened or lengthened, thereby causing contraction and relaxation of the entire muscle. An increase in myofibril size means an increase in myofiber thickness, resulting in an increase in muscle.

Types of myofibers that make up muscles are mainly classified into type I, type IIA, and type IIB depending on the metabolic process that produces ATP and the contraction velocity. “Type I myofibers” have a slow contraction velocity and contain a large number of myoglobin and mitochondria, which makes them suitable for sustained, low-intensity aerobic activity. Type I myofibers are also called red muscle because they have a red color, and soleus typically belongs thereto. On the other hand, “type IIB myofibers” have a fast contraction velocity and thus are used for very short but high-intensity anaerobic exercise. The type IIB myofibers have a white color due to a low content of myoglobin. “Type IIA myofibers” have the intermediate characteristics between the above-mentioned two myofibers. As aging progresses, not only the composition of type I and II myofibers vary depending on muscle area, but also all types of myofibers decrease.

Skeletal muscles have characteristics of being regenerated and maintained according to the environment, but these characteristics are lost with age. As a result, as aging progresses, not only muscle mass decreases but muscle strength is also lost. As a signal transduction system involved in muscle growth and regeneration, there is signal transduction which is mediated by insulin-like growth factor 1 (IGF-1)/AKT and regulates protein synthesis. Activation of IGF-1 receptor (IGF-1R) present in the muscle cell membrane increases AKT phosphorylation through IRS1 and PI3K phosphorylation, and the latter activates mTORC phosphorylation. Activation of mTORC increases phosphorylation of ribosomal protein S6 kinase beta-1 (p70S6K1), which increases mRNA translation while, at the same time, increasing activity of eukaryotic translation initiation factor 4G (eIF4G) and phosphorylating eukaryotic translation initiation factor 4E binding protein 1 (4E-BP1) proteins. eIF4G and 4E-BP1 are involved in forming an eIF4F complex. That is, eIF4G binds to eIF4A and eIF4E, to form the eIF4F complex. On the other hand, phosphorylation of 4E-BP1 inhibits its ability to bind to eIF4E, leading to an increase in free eIF4E. The latter binds to other translation initiation factors (eIF4G and eIF4A), to form an eIF4F complex, and the thus formed eIF4F complex stabilizes the ribosomal structure, thereby promoting translation initiation and ultimately increasing protein synthesis.

In addition, AKT phosphorylation promotes myofiber growth by increasing expression of eIF2B through glycogen synthase kinase 3 (GSK3), and also inhibits muscle loss by inhibiting expression of forkhead box O (FOXO), a protein degradation-related transcription factor. Muscle loss is regulated by signal transduction which is mediated by receptors of the TGF-β family, including myostatin, transforming growth factor beta (TGF-β), and activin. Binding of a ligand to the TGF-β type II receptor phosphorylates the type I receptor. The latter phosphorylates the smad 2/3 complex, and eventually activates FOXO. The latter increases gene expression of the muscle-specific ubiquitin-ligase, muscle RING-finger protein-1 (MURF1), and muscle atrophy F-box (MAFbx)/Atrogin-1, which attaches ubiquitin to the lysine site on a target protein so that protein degradation is promoted, and eventually muscle decrease is induced.

### SUMMARY

The present disclosure is intended to provide a pharmaceutical composition for preventing or treating a muscle disease, comprising, as an active ingredient, azelaic acid or a pharmaceutically acceptable salt thereof, and the like.

However, the technical problem to be achieved by the present disclosure is not limited to the above-mentioned problems, and other problems not mentioned will be clearly understood by those skilled in the art from the following description.

The present disclosure provides a pharmaceutical composition for preventing or treating a muscle disease, comprising, as an active ingredient, azelaic acid or a pharmaceutically acceptable salt thereof.

The azelaic acid or a pharmaceutically acceptable salt thereof may increase expression of p-4E-BP1 and p-p70S6K1 proteins.

The azelaic acid or a pharmaceutically acceptable salt thereof may decrease expression of muscle RING-finger protein (MuRF1), muscle atrophy F-box (MaFbx), or myostatin.

The muscle disease may be a muscle disease caused by muscle function decline, muscle decrease, muscle atrophy, muscle wasting, or muscle degeneration.

The muscle disease may be any one or more selected from the group consisting of atony, muscular atrophy, muscular dystrophy, myasthenia, cachexia, rigid spine syndrome, amyotrophic lateral sclerosis (Lou Gehrig's disease), Charco-Marie-Tooth disease, and sarcopenia.

In addition, the present disclosure provides a pharmaceutical composition for promoting muscle differentiation, regenerating muscles, or enhancing muscles, comprising, as an active ingredient, azelaic acid or a pharmaceutically acceptable salt thereof.

In addition, the present disclosure provides a health functional food composition for promoting muscle differentiation, regenerating muscles, or enhancing muscles, comprising, as an active ingredient, azelaic acid or a pharmaceutically acceptable salt thereof.

In addition, the present disclosure provides a pharmaceutical composition for increasing muscle mass or promoting muscle production, comprising, as an active ingredient, azelaic acid or a pharmaceutically acceptable salt thereof.

In addition, the present disclosure provides a health functional food composition for increasing muscle mass or promoting muscle production, comprising, as an active ingredient, azelaic acid or a pharmaceutically acceptable salt thereof.

In addition, the present disclosure provides a health functional food composition for improving muscle function, comprising, as an active ingredient, azelaic acid or a pharmaceutically acceptable salt thereof.

In addition, the present disclosure provides a cosmetic composition for improving muscle function, comprising, as an active ingredient, azelaic acid or a pharmaceutically acceptable salt thereof.

The present disclosure relates to a composition for preventing or treating a muscle disease, or improving muscle function, comprising, as an active ingredient, azelaic acid or a pharmaceutically acceptable salt thereof, wherein the azelaic acid may be used for preventing or treating a muscle disease, differentiating muscles, regenerating muscles and enhancing muscles, or for increasing muscle mass, promoting muscle production, or improving muscle function in view of the fact that in muscle cells, the azelaic acid can increase expression of proteins associated with muscle protein synthesis and muscle mass increase and can inhibit expression of enzymes involved in muscle protein degradation at their mRNA level; and therefore, the azelaic acid can exhibit a muscle strength-enhancing effect through muscle differentiation, muscle regeneration, and muscle mass increase in a muscle disease caused by muscle function decline, muscle wasting, or muscle degeneration, and can inhibit muscle decrease.

### BRIEF DESCRIPTION OF THE DRAWINGS

FIGS. 1A-1B and FIGS. 2A-2B illustrate results obtained by analyzing changes in myotube length in mouse myoblasts (p<0.05).

FIG. 3A and FIG. 3B illustrate results obtained by identifying changes in mRNA expression level (FIG. 3A) and protein expression level (FIG. 3B) of protein degradation- and synthesis-related molecules in mouse myoblasts treated with azelaic acid.

### DESCRIPTION OF THE PREFERRED EMBODIMENTS

The present inventors have identified that azelaic acid, a natural product having few side effects, is effective in muscle potentiation and muscle loss improvement by inhibiting degradation of muscle proteins and promoting synthesis thereof, and thus have completed the present disclosure.

Hereinafter, the terms used in the present disclosure are described.

As used herein, the term “myo” comprehensively refers to sinew, muscle, and tendon; and the term “muscle function” means an ability to exert force through contraction of muscles and includes muscle strength which is the muscle's ability to exert maximum contraction to withstand resistance, muscular endurance which is an ability to indicate how long or how many times muscles can repeat contraction and relaxation against a given weight, and power which is an ability to exert strong force in a short time. This muscle function is proportional to muscle mass, and the term “improving muscle function” means making muscle function better.

Azelaic acid has Cas No. 123-99-9, has a molecular formula of C9H16O4, has a molecular weight of 188.22 g/mol, and has a structure represented by the following Formula 1. Azelaic acid has IUPAC name of nonanedioic acid and is called by other names such as nonanedioic ACID; Finacea; anchoic acid. Azelaic acid is a white solid and is soluble in ethanol and water.

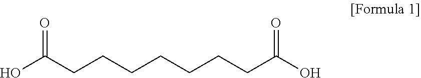 [SMILES: O=C(O)CCCCCCCC(=O)O]

Azelaic acid is known to exist in Solanum tuberosum (potato, leaves), Phaseolus vulgaris ‘Black turtle (black bean, roots), or the like.

Azelaic acid is listed, as a flavoring material, in the Food Additive Database in COE (Council of Europe), KFDA (Korea Food and Drug Administration), and FDA (U.S. Food & Drug Administration), and is used as a supplement or the like.

To date, azelaic acid has been reported to have antioxidant activity as physiological activity. It has been reported that azelaic acid exhibits an antioxidant effect by inhibiting the reaction of an enzyme such as nicotinamide adenine dinucleotide phosphate (NADP) oxidase. Azelaic acid has another physiological activity which is anti-inflammation, and such physiological activity has been demonstrated through inhibition of mRNA expression and protein secretion of interleukin-1β, interleukin-6, and TNF-α by UVB in a case where human epidermal cells are treated with 20 mM azelaic acid. It has been reported that azelaic acid has antimicrobial activity, in which an ex vivo experiment was conducted on viability of various strains of skin microorganisms in 0.5 mol/l (8.4% w/v) azelaic acid solution, and as a result, all bacterial strains showed at least 40-fold decreased viability for 24 hours, through which a bactericidal effect of azelaic acid has been demonstrated.

Meanwhile, in a toxicity test of azelaic acid, azelaic acid was orally administered to rats at 10,000, 14,000, 16,800, 19,600, and 25,000 mg/kg, respectively. As a result, it has been reported that azelaic acid has an LD₅₀ value of 15,800 mg/kg.

Hereinafter, the present disclosure will be described in detail.

Pharmaceutical Compositions for Preventing or Treating Muscle Disease/Promoting Muscle Differentiation, Regenerating Muscles, or Enhancing Muscles/Increasing Muscle Mass or Promoting Muscle Production

The present disclosure provides a pharmaceutical composition for preventing or treating a muscle disease, comprising, as an active ingredient, azelaic acid or a pharmaceutically acceptable salt thereof.

In addition, the present disclosure provides a pharmaceutical composition for promoting muscle differentiation, regenerating muscles, or enhancing muscles, comprising, as an active ingredient, azelaic acid or a pharmaceutically acceptable salt thereof.

In addition, the present disclosure provides a pharmaceutical composition for increasing muscle mass or promoting muscle production, comprising, as an active ingredient, azelaic acid or a pharmaceutically acceptable salt thereof.

In the pharmaceutical composition of the present disclosure, the azelaic acid is preferably a compound having a structure of the following Formula 1, but is not limited thereto; and any isomer, hydrate, or derivative within a scope that could be understood by one skilled in the art to have the same or similar activity as azelaic acid is applicable:

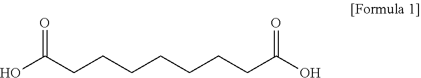 [SMILES: O=C(O)CCCCCCCC(=O)O]

A method for obtaining the azelaic acid is not particularly limited, and one which has been isolated from a plant containing the azelaic acid, has been chemically synthesized using a known preparation method, or is commercially available may be used.

In the pharmaceutical composition of the present disclosure, the azelaic acid or a pharmaceutically acceptable salt thereof may increase expression of p-4E-BP1 and p-p70S6K1 proteins, and may decrease expression of muscle RING-finger protein (MuRF1), muscle atrophy F-box (MaFbx), or myostatin.

Specifically, representative molecules related to protein synthesis include p70S6K1, 4E-BP1, and eIF members, and activity of these three molecules is regulated by mTORC which is at an upper level. Activation of mTORc phosphorylates p70S6K1 and activated p70S6K1 phosphorylates 40S ribosomal protein S6, thereby increasing mRNA translation. Activation of mTORC also increases activity of eIF4G and at the same time, phosphorylates 4E-BP1, these two molecules being involved in forming an eIF4F complex. That is, eIF4G binds to eIF4A and eIF4E, to form the eIF4F complex. On the other hand, phosphorylation of 4E-BP1 inhibits its ability to bind to eIF4E, leading to an increase in free eIF4E. The latter binds to other translation initiation factors (eIF4G and eIF4A), to form an eIF4F complex, and the thus formed eIF4F complex stabilizes the ribosomal structure, thereby promoting translation initiation and ultimately increasing protein synthesis. Muscle atrophy F-box (MAFbx)/Atrogin-1 and muscle RING finger 1 (MuRF1) are muscle-specific ubiquitin-ligases and are representative proteins that cause ubiquitin to be attached to the lysine site in a target protein so that protein degradation is promoted and ultimately muscle decrease is induced. The composition of the present disclosure may decrease expression of muscle RING finger 1 (MuRF1) and muscle atrophy F-box (MAFbx), thereby inhibiting muscle decrease.

In the pharmaceutical composition of the present disclosure, the muscle disease includes a range of diseases caused by muscle function decline, muscle decrease, muscle atrophy, muscle wasting, or muscle degeneration. Specifically, the muscle disease is preferably, but is not limited to, any one or more selected from the group consisting of atony, muscular atrophy, muscular dystrophy, myasthenia, cachexia, rigid spine syndrome, amyotrophic lateral sclerosis (Lou Gehrig's disease), Charco-Marie-Tooth disease, and sarcopenia. In addition, the muscle wasting or the muscle degeneration occurs due to genetic factors, acquired factors, aging, or the like. The muscle wasting is characterized by a gradual loss of muscle mass, and weakening and degeneration of muscles, in particular, skeletal or voluntary muscles and heart muscles.

In addition, in view of using the pharmaceutical composition of the present disclosure for promoting muscle differentiation, regenerating muscle or enhancing muscles, muscle cell differentiation means inducing a muscle developmental program that specifies components of myofibers such as contractile organelle (myofibril). Useful therapeutic agents for differentiation may increase amounts of all myofiber components in diseased tissues, by at least about 10%, more preferably at least 50%, and most preferably at least 100%, as compared with equivalent tissues in similarly treated control animals.

In addition, in view of using the pharmaceutical composition of the present disclosure for promoting muscle differentiation, regenerating muscles, or enhancing muscles, or for increasing muscle mass, muscle growth may occur due to an increase in fiber size and/or an increase in number of fibers. The muscle growth can be measured by A) increase in wet weight, B) increase in protein content, C) increase in number of myofibers, and D) increase in diameter of myofibers. An increase in myofiber growth may be defined as an increase in diameter in a case where the diameter is defined as the minor axis of sectional ellipsoid. Useful therapeutic agents are those which increase wet weight, protein content, and/or diameter, by at least 10%, more preferably at least 50%, and most preferably at least 100%, in animals having at least about 10% muscle degeneration as compared with previously similarly treated control animals (that is, animals having degenerated muscle tissues, not treated with compounds for muscle growth). Compounds that increase growth by increasing the number of myofibers are useful as therapeutic agents in a case where the compounds increase the number of myofibers in diseased tissues by at least 1%, more preferably at least 20%, and most preferably at least 50%. These percentage values are relatively determined with respect to the basal level in untreated, unaffected comparative mammals or contralateral unaffected muscles, in a case where the compounds are administered and act locally.

In addition, in view of using the pharmaceutical composition of the present disclosure for promoting muscle differentiation, regenerating muscles, or enhancing muscles, regenerating muscles means a process in which new myofibers are generated from myoblasts. As described above, useful therapeutic agents for regeneration increase the number of new fibers by at least about 1%, more preferably at least 20%, and most preferably at least 50%.

Muscle cell differentiation means inducing a muscle developmental program that specifies components of myofibers such as contractile organelle (myofibril). Useful therapeutic agents for differentiation may increase amounts of all myofiber components in diseased tissues, by at least about 10%, more preferably at least 50%, and most preferably at least 100%, as compared with equivalent tissues in similarly treated control animals.

In addition, in view of using the pharmaceutical composition of the present disclosure for increasing muscle mass or promoting muscle production, “increasing muscle mass” is to improve growth, particularly, of muscles, among body components. Muscle mass can be increased through physical exercise and endurance improvement, and can be increased by administering to the body a substance having a muscle-increasing effect; and there is no limitation on types of muscles.

In the pharmaceutical composition of the present disclosure, the amount of azelaic acid or a pharmaceutically acceptable salt thereof may be contained in the composition at a concentration of 0.1 μM to 1,000 μM. However, the concentration is not limited thereto. Here, in a case where azelaic acid is less than the concentration range, there is a problem that it is difficult to exert an effect of preventing or treating a muscle disease due to decreased protein synthesis and degradation activity in muscle cells. In a case where azelaic acid is greater than the concentration range, there may be a concern about toxicity including cytotoxicity.

The azelaic acid of the present disclosure may be used in the form of a pharmaceutically acceptable salt. As the salt, an acid addition salt formed with a pharmaceutically acceptable free acid is useful. The acid addition salt is obtained from inorganic acids such as hydrochloric acid, nitric acid, phosphoric acid, sulfuric acid, hydrobromic acid, hydroiodic acid, nitrous acid, or phosphorous acid, and non-toxic organic acids such as aliphatic mono- and dicarboxylates, phenyl-substituted alkanoates, hydroxy alkanoates and alkanedioates, aromatic acids, aliphatic and aromatic sulfonic acids. Such pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, or mandelate.

The acid addition salt according to the present disclosure may be prepared by a conventional method, for example, by dissolving the azelaic acid in an excess of aqueous acid solution and precipitating the salt using a water-miscible organic solvent such as methanol, ethanol, acetone, or acetonitrile. The acid addition salt may also be prepared by heating the equal amounts of azelaic acid and acid or alcohol in water, and then evaporating the mixture to dryness or suction-filtering the precipitated salt.

In addition, bases may be used to make pharmaceutically acceptable metal salts. Alkali metal or alkaline earth metal salts are obtained, for example, by dissolving compounds in an excess of alkali metal hydroxide or alkaline earth metal hydroxide solution, filtering undissolved compound salts, and evaporating the filtrate to dryness. Here, as the metal salts, it is pharmaceutically suitable to prepare sodium, potassium, or calcium salts. In addition, silver salts corresponding thereto are obtained by reacting alkali metal or alkaline earth metal salts with a suitable silver salt (for example, silver nitrate). In addition, the azelaic acid of the present disclosure includes not only pharmaceutically acceptable salts, but also all salts, hydrates, and solvates which can be prepared by conventional methods.

The addition salt according to the present disclosure may be prepared by a conventional method, for example, by dissolving azelaic acid in a water-miscible organic solvent such as acetone, methanol, ethanol, or acetonitrile, adding thereto an excess of organic acid or aqueous inorganic acid solution, and then performing precipitation or crystallization. Subsequently, the addition salt may be obtained by evaporating the solvent or excess acid in this mixture and then performing drying, or may be prepared by suction-filtering the precipitated salt.

The pharmaceutical composition of the present disclosure may be in various oral or parenteral preparations. In a case of being formulated into the preparation, preparation may be carried out using one or more of buffers (for example, saline or PBS), antioxidants, bacteriostatic agents, chelating agents (for example, EDTA or glutathione), fillers, extenders, binders, adjuvants (for example, aluminum hydroxide), suspending agents, thickeners, wetting agents, disintegrants or surfactants, diluents or excipients.

Solid preparations for oral administration include tablets, pills, powders, granules, capsules, and the like. Such solid preparations may be prepared by mixing one or more compounds with at least one excipient such as starch (including corn starch, wheat starch, rice starch, potato starch, and the like), calcium carbonate, sucrose, lactose, dextrose, sorbitol, mannitol, xylitol, erythritol, maltitol, cellulose, methyl cellulose, sodium carboxymethyl cellulose, hydroxypropyl methylcellulose, and gelatin. For example, tablets or sugarcoated tablets may be obtained by blending an active ingredient with a solid excipient, grinding the blend, adding a suitable adjuvant thereto, and then processing the resultant into a granule mixture.

In addition to simple excipients, lubricants such as magnesium stearate and talc are also used. Liquid preparations for oral administration include suspensions, solutions, emulsions, syrups, and the like. In addition to water or liquid paraffin, which is a commonly used simple diluent, the liquid preparations may contain various excipients such as a wetting agent, a sweetener, a fragrance, and a preservative. In addition, in some cases, crosslinked polyvinylpyrrolidone, agar, alginic acid, sodium alginate, or the like may be added as a disintegrant. An anticoagulant, a lubricant, a wetting agent, a flavoring agent, an emulsifying agent, an antiseptic agent, or the like may be further contained.

Preparations for parenteral administration include sterile aqueous solutions, non-aqueous solvents, suspensions, emulsions, lyophilized preparations, and suppositories. For the non-aqueous solvents and suspensions, propylene glycol, polyethylene glycol, vegetable oil such as olive oil, injectable ester such as ethyl oleate, and the like can be used. As bases of the suppository, witepsol, macrogol, Tween 61, cacao butter, laurin butter, glycerol, gelatin, or the like may be used.

The pharmaceutical composition of the present disclosure may be administered orally or parenterally. In a case of being administered parenterally, the pharmaceutical composition may be formulated, according to methods known in the art, in the form of an external preparation for skin; an injection to be injected intraperitoneally, rectally, intravenously, intramuscularly, subcutaneously, intrauterine epidurally, or intracerebrovascularly; a preparation for transdermal administration; or a nasal inhalant.

The injection must be sterilized and protected against contamination of microorganisms such as bacteria and fungi. In a case of the injection, examples of suitable carriers may include, but are not limited to, solvents or dispersion media which contain water, ethanol, polyol (for example, glycerol, propylene glycol, and liquid polyethylene glycol), mixtures thereof, and/or vegetable oil. More preferably, as the suitable carriers, an isotonic solution such as Hank's solution, Ringer's solution, triethanolamine-containing phosphate buffered saline (PBS) or sterilized water for injection, 10% ethanol, 40% propylene glycol, and 5% dextrose, or the like may be used. In order to protect the injection against contamination of microorganisms, various antibacterial agents and antifungal agents such as paraben, chlorobutanol, phenol, sorbic acid, and thimerosal may be further contained. In addition, in most cases, the injection may further contain an isotonic agent such as sugar or sodium chloride.

The preparation for transdermal administration takes forms such as an ointment, a cream, a lotion, a gel, a liquid for external use, a paste, a liniment, and an aerosol. In this case, “transdermal administration” means administering a pharmaceutical composition topically to skin so that an effective amount of an active ingredient contained in the pharmaceutical composition is delivered into the skin.

In a case of a preparation for inhalation administration, a compound to be used according to the present disclosure may be conveniently delivered in the form of an aerosol spray from a pressurized pack or a nebulizer, using a suitable propellant such as dichlorofluoromethane, trichlorofluoromethane, dichlorotetrafluoroethane, carbon dioxide, or another suitable gas. In a case of a pressurized aerosol, a unit dosage may be determined by providing a valve that delivers a metered amount. For example, gelatin capsules and cartridges for use in an inhaler or insufflator may be formulated to contain a powder mixture of a compound and a suitable powder base such as lactose and starch. Preparations for parenteral administration are described in Remington's Pharmaceutical Science, 15th Edition, 1975. Mack Publishing Company, Easton, Pa. 18042, Chapter 87: Blaug, Seymour, which is a prescription manual commonly known in all pharmaceutical chemistries.

The pharmaceutical composition of the present disclosure is administered in a pharmaceutically effective amount. In the present disclosure, the term “pharmaceutically effective amount” means an amount which is sufficient to treat a disease at a reasonable benefit/risk ratio applicable to medical treatment, and an effective dose level can be determined depending on factors including type, severity of the patient's disease, activity of the drug, sensitivity to the drug, time of administration, route of administration and rate of excretion, duration of treatment, the simultaneously used drug, and other factors well known in the medical field. The pharmaceutical composition of the present disclosure may be administered as an individual therapeutic agent or in combination with another therapeutic agent. The pharmaceutical composition may be administered sequentially or simultaneously with a conventional therapeutic agent, and may be administered in single or multiple doses. That is, a total effective amount of the pharmaceutical composition of the present disclosure may be administered to a patient as a single dose, or as multiple doses by a fractionated treatment protocol intended for a long-term administration. It is important to administer an amount such that a maximum effect can be obtained with a minimum amount without side effects by taking all of the above-described factors into consideration, and such an amount can be readily determined by those skilled in the art.

The dose of the pharmaceutical composition of the present disclosure varies depending on the patient's body weight, age, sex, health condition, diet, time of administration, mode of administration, excretion rate and severity of the disease. For a daily dose, the pharmaceutical composition may be administered as a single dose or in several divided doses such that azelaic acid is administered in an amount of preferably 0.01 to 50 mg and more preferably 0.1 to 30 mg per kg body weight a day in a case of parenteral administration, and such that azelaic acid is administered in an amount of preferably 0.01 to 100 mg and more preferably 0.01 to 10 mg per kg body weight a day in a case of oral administration. However, since the dose may be increased or decreased depending on route of administration, severity of obesity, sex, body weight, age, and the like, such a dose does not limit the scope of the present disclosure in any way.

The pharmaceutical composition of the present disclosure may be used either alone or in combination with methods which use surgery, radiation therapy, hormonal therapy, chemotherapy, or biological response modifiers.

The pharmaceutical composition of the present disclosure may also be provided as a preparation for external use, comprising azelaic acid as an active ingredient. In a case where the pharmaceutical composition of the present disclosure for preventing or treating a muscle disease is used as an external preparation for skin, the pharmaceutical composition may further contain adjuvants commonly used in the field of dermatology such as any other ingredients commonly used for the external preparation for skin including a fatty substance, an organic solvent, a solubilizing agent, a concentrating agent and a gelling agent, a softening agent, an antioxidant, a suspending agent, a stabilizing agent, a foaming agent, a fragrance, a surfactant, water, an ionic emulsifying agent, a nonionic emulsifying agent, a filling agent, a metal ion blocking agent, a chelating agent, a preservative, a vitamin, a blocking agent, a wetting agent, essential oil, a dye, a pigment, a hydrophilic activator, a lipophilic activator, a lipid vesicle, and the like. In addition, the above ingredients may be introduced in an amount commonly used in the field of dermatology.

In a case where the pharmaceutical composition of the present disclosure for preventing or treating a muscle disease is provided as an external preparation for skin, the pharmaceutical composition may be, but is not limited to, a preparation such as an ointment, a patch, a gel, a cream, and a spray.

Health Functional Food Compositions for Preventing or Improving Muscle Disease/for Promoting Muscle Differentiation, Regenerating Muscles, or Enhancing Muscles/for Increasing Muscle Mass or Promoting Muscle Production

In addition, the present disclosure provides a health functional food composition for preventing or improving a muscle disease, comprising, as an active ingredient, azelaic acid or a pharmaceutically acceptable salt thereof.

In addition, the present disclosure provides a health functional food composition for promoting muscle differentiation, regenerating muscles, or enhancing muscles, comprising, as an active ingredient, azelaic acid or a pharmaceutically acceptable salt thereof.

In addition, the present disclosure provides a health functional food composition for increasing muscle mass or promoting muscle production, comprising, as an active ingredient, azelaic acid or a pharmaceutically acceptable salt thereof.

In addition, the present disclosure provides a health functional food composition for improving muscle function, comprising, as an active ingredient, azelaic acid or a pharmaceutically acceptable salt thereof.

In the health functional food composition, the details of the azelaic acid are as described above.

In the health functional food compositions for preventing or improving a muscle disease/for promoting muscle differentiation, regenerating muscles, or enhancing muscles/for increasing muscle mass or promoting muscle production according to the present disclosure, in a case where the azelaic acid is used as an additive for the health functional food, the azelaic acid may be added as it is or used together with other foods or food ingredients, and may be appropriately used according to a conventional method. A mixed amount of active ingredients can be appropriately determined depending on each intended purpose of use, such as prophylactic, health, or therapeutic purpose.

Preparations for the health functional food may be in any form including powders, granules, pills, tablets, capsules, as well as general foods or beverages.

Types of the food are not particularly limited. The examples thereof to which the above substance can be added may include meats, sausages, bread, chocolates, candies, snacks, confections, pizza, ramen, other noodles, gums, dairy products including ice cream, various soups, beverages, tea, drinks, alcoholic beverages, and vitamin complexes, and may include all foods in a usual sense.

In general, at the time of manufacturing foods or beverages, the azelaic acid may be added in an amount of 15 parts by weight or less and preferably 10 parts by weight or less with respect to 100 parts by weight of the raw material. However, in a case of long-term intake for health and hygiene or for health control, the amount may be less than the above range, and the azelaic acid can also be used in an amount which is more than the above range because the present disclosure has no problem in terms of safety from the viewpoint that fractions from natural products are used.

Among the health functional foods according to the present disclosure, the beverage may contain, as additional ingredients, various flavoring agents, natural carbohydrates, or the like as in ordinary beverages. The above-mentioned natural carbohydrate may be a monosaccharide such as glucose and fructose; a disaccharide such as maltose and sucrose; a polysaccharide such as dextrin and cyclodextrin; or sugar alcohol such as xylitol, sorbitol, and erythritol. As the sweetening agent, a natural sweetening agent such as thaumatin and a stevia extract; a synthetic sweetening agent such as saccharin and aspartame, or the like may be used. A proportion of the natural carbohydrate may be about 0.01 to 0.04 g and preferably about 0.02 to 0.03 g, per 100 mL of the beverage according to the present disclosure.

In addition to the above-mentioned ingredients, the health food composition for promoting muscle differentiation, regenerating muscles, or enhancing muscles according to the present disclosure may further contain various nutrients, vitamins, electrolytes, flavoring agents, colorants, pectic acid, salts of pectic acid, alginic acid, salts of alginic acid, organic acids, protective colloids, thickeners, pH adjusting agents, stabilizers, preservatives, glycerin, alcohol, carbonating agents used in carbonated beverages. In addition, the health food composition for promoting muscle differentiation, regenerating muscles, or enhancing muscles according to the present disclosure may further contain fruit flesh for preparing a natural fruit juice, a fruit juice beverage, or a vegetable beverage. These ingredients may be used independently or in admixture. Proportions of such additives are not limited, and are generally selected in a range of 0.01 to 0.1 parts by weight per 100 parts by weight of the health functional food of the present disclosure.

Cosmetic Compositions for Improving Muscle Function

In addition, the present disclosure provides a cosmetic composition for improving muscle function, comprising, as an active ingredient, azelaic acid or a pharmaceutically acceptable salt thereof. The cosmetic composition is not particularly limited, and may be used for external use on skin or may be ingested orally.

The composition for improving muscle function of the present disclosure may also be a cosmetic composition. The cosmetic composition of the present disclosure contains azelaic acid as an active ingredient, and may be prepared, together with dermatologically acceptable excipients, in the form of basic cosmetic compositions (face cleansing agents such as lotion, cream, essence, cleansing foam, and cleansing water, pack, and body oil), color cosmetic compositions (foundation, lipstick, mascara, and makeup base), hair product compositions (shampoo, rinse, hair conditioner, and hair gel), soaps, and the like.

Examples of such excipients may include, but are not limited to, skin emollients, skin penetration enhancers, colorants, fragrances, emulsifiers, concentrating agents, and solvents. In addition, flavoring agents, pigments, bactericides, antioxidants, preservatives, moisturizers, and the like may be further contained. For the purpose of improving physical properties, thickeners, inorganic salts, synthetic polymeric substances, and the like may be further contained. For example, in a case where a face cleansing agent and a soap are prepared with the cosmetic composition of the present disclosure, preparation can be easily carried out by adding the azelaic acid to common bases for the face cleansing agent and the soap. In a case of preparing the cream, preparation may be carried out by adding azelaic acid or a salt thereof to a typical oil-in-water (O/W) cream base. To this may be further added a flavoring agent, a chelating agent, a pigment, an antioxidant, a preservative, and the like as well as a synthetic or natural material, such as a protein, a mineral, and a vitamin, which is intended to improve physical properties.

A content of azelaic acid contained in the cosmetic composition of the present disclosure is, but is not limited to, preferably 0.001% to 10% by weight, and more preferably 0.01% to 5% by weight, with respect to a total weight of the entire composition. In a case where the content is less than 0.001% by weight, a desired anti-aging or wrinkle-improving effect cannot be expected. In a case where the content is more than 10% by weight, it may be difficult to maintain safety or to formulate preparations.

As described above, the composition comprising, as an active ingredient, azelaic acid or a pharmaceutically acceptable salt thereof of the present disclosure can be used for preventing or treating a muscle disease, differentiating muscles, regenerating muscles and increasing muscle mass, or improving muscle function in view of the fact that in myoblasts, the azelaic acid increases 4E-BP1 and p70S6K1 protein phosphorylation and inhibits gene expression of MuRF1 and MaFbx/Atrogin1, thereby exhibiting a muscle strength-enhancing effect through muscle differentiation, muscle regeneration, and muscle mass increase in a muscle disease caused by muscle function decline, muscle wasting, or muscle degeneration, and inhibiting muscle decrease.

Hereinafter, preferred examples are given to help understand the present disclosure. However, the following examples are merely provided to more easily understand the present disclosure, and the scope of the present disclosure is not limited by the following examples.

### EXAMPLES

### Analysis of Muscle-Potentiating and Muscle Loss-Inhibitory Efficacy of Azelaic Acid using Mouse Myoblasts

### Example 1

### Identification of Changes in Myotube Length

1-1. Experiment Method

1) Cell Culture

Mouse myoblast cell line (C2C12 cells) was purchased from ATCC (Manassas, Va., USA) and used. The purchased cell line was incubated in an incubator at 37° C., 5% CO₂ using 10% fetal bovine serum medium (Gibco-BRL) and used for experiments. When the cell line reached a confluency of about 80%, the cells were differentiated into myotubes using 2% horse serum medium (Gibco-BRL). In order to identify muscle-potentiating efficacy, treatment with 100 μM azelaic acid (CAS Number 123-99-9, Sigma) was performed for two days from the day when differentiation began, and a control was treated with 100 μM dimethyl sulfoxide (DMSO; Sigma). In order to identify muscle loss-inhibitory efficacy, combined treatment with 50 μM dexamethasone (dexa; Sigma) and 100 μM azelaic acid (CAS Number 123-99-9, Sigma) was performed for two days from the 4^{th} day of differentiation.

2) Wright-Giemsa Staining

The cells were washed twice with phosphate buffered saline (PBS), and then fixed for 10 minutes with 100% methanol. After the fixation was completed, the resultant was naturally dried at room temperature for 10 minutes, and then stained for 30 minutes by adding dropwise a Wright-Giemsa staining solution (ASAN Pharm. Co., Ltd., Seoul, South Korea) that specifically stains myotubes.

3) Measurement of Myotube Length

Stained myotubes were photographed at ×40 magnification using a fluorescence microscope (IX 71, Olympus) and analyzed using Image J software (USA). Six sections of each well were randomly selected and micrographed. Lengths of at least 100 myotubes from each well were analyzed (3 repetitions/group).

1-2. Experiment Results

1) Identification of Changes in Myotube Length

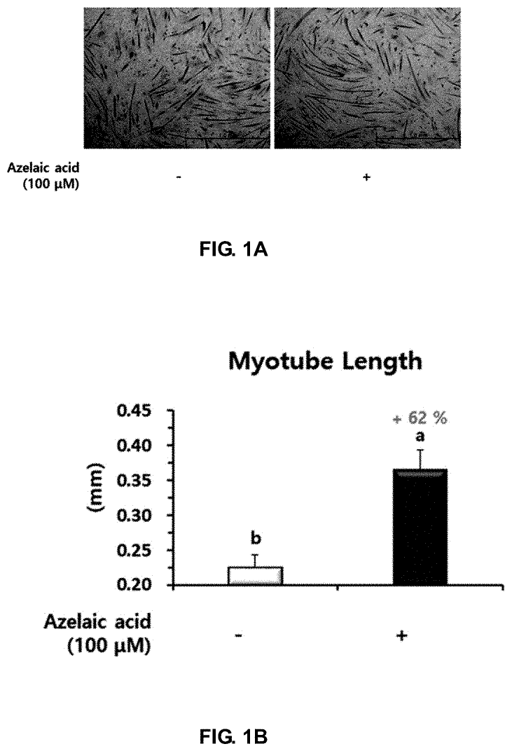

In order to identify muscle-potentiating efficacy of azelaic acid on mouse myoblasts, myotubes were specifically stained using a Giemsa solution, and then visualized. As a result, it was identified that treatment with azelaic acid causes a significant 62% increase in myotube length (FIGS. 1A-1B). Values represent the mean±SEM of 3 experiments, and values marked with different letters indicate statistical significance (P<0.05).

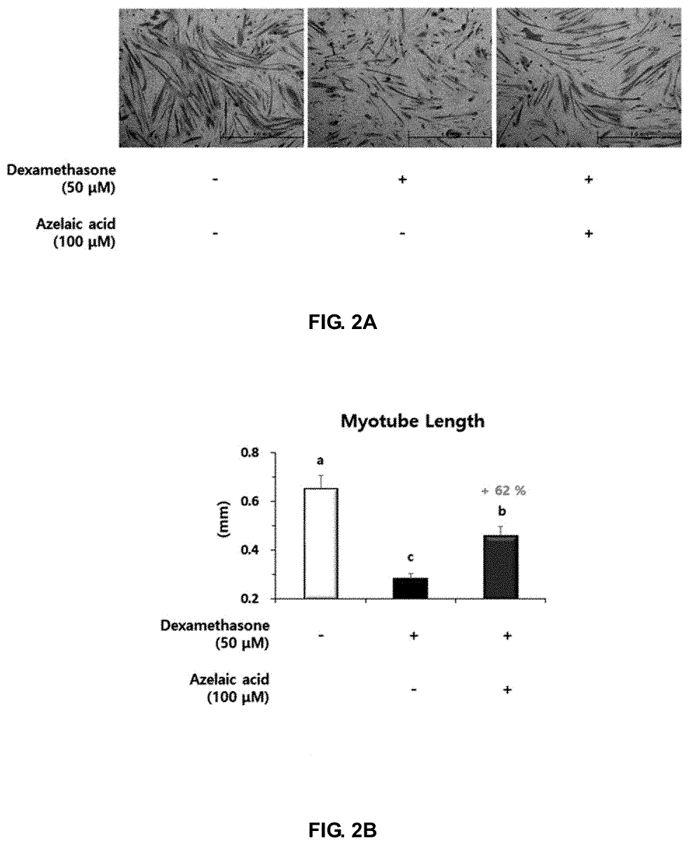

Subsequently, in order to identify muscle loss-inhibitory efficacy of azelaic acid on mouse myoblasts, myotubes were specifically stained using a Giemsa solution, and then visualized. As a result, it was identified that treatment with dexamethasone causes a significant decrease in myotube length, and it was identified that treatment with azelaic acid causes a significant 62% increase in the myotube length which has decreased due to dexamethasone (FIGS. 2A-2B). Therefore, it can be seen that azelaic acid increases myotube length in mouse myoblasts, thereby promoting muscle growth and inhibiting muscle loss. Values represent the mean±SEM of 3 experiments, and values marked with different letters indicate statistical significance (P<0.05).

### Example 2

### Elucidation of Action Mechanism

2-1. Experiment Method

1) RNA Isolation using Trizol Method and Real-Time (RT) PCR (Quantitative Reverse-Transcription Polymerase Chain Reaction)

334 μl of Trizol solution was added per 1×10⁷ cells of mouse myoblasts, and grinding was performed. Then, the resultant was centrifuged at 4° C. and 12,000×g for 10 minutes. The supernatant was transferred to a new tube. Then, 67 μl of chloroform was added thereto and vortexing was performed. Again, the supernatant was transferred to a new tube, and isopropanol was added so that a ratio of the supernatant to isopropanol was 1:1. After performing vigorous shaking 10 times, the tube was allowed to stand at room temperature for 15 minutes, and centrifuged at 12,000×g and 4° C. for 10 minutes. Then, the supernatant was removed, and 1 mL of 70% ethanol was added to the remaining precipitate. Then, the resultant was centrifuged at 7,500×g and 4° C. for 5 minutes. After removing ethanol, the tube containing an RNA precipitate was dried at room temperature for 15 minutes, and the RNA pellet was dissolved using nuclease-free water. A UV/VIS spectrophotometer (Beckman Coulter, DU730) was used to measure a concentration of the extracted RNA sample at wavelengths of 260 nm and 280 nm. Agarose gel electrophoresis was performed to check integrity of the RNA sample.

On the RNA sample extracted from the mouse myoblasts, reverse transcription was performed using oligo dT primers and superscript reverse transcriptase (GIBCO BRL, Gaithersburg, Md., USA), to synthesize cDNA. Using, as a template, cDNA obtained through the reverse transcription and, as primers, 5′ and 3′ flanking sequences of cDNA of the gene to be amplified, quantitative PCR was performed with iQ SYBR green supermix (Bio-Rad) and CFX Connect™ Real-Time PCR Detection System (Bio-Rad). The primer sequences used at this time are shown in Table 1.

TABLE 1

| | | | Annea- | |
|---|---|---|---|---|
| | | | ling | PCR |
| | | | temper- | pro- |
| Gene | | | ature | duct |
| description | Primers | Sequences (5′→3′) | (° C.) | (bp) |
| MAFbx | F | GTCCAGAGAGTCGGCAAGTC | 63 | 141 |
| (synonym: | R | GTCGGTGATCGTGAGACCTT | | |
| Atrogin-1) | | | | |
| MuRF1 | F | ACATCTACTGTCTCACGTGT | 58 | 106 |
| (synonym: | R | TGTCCTTGGAAGATGCTTTG | | |
| TRAM63) | | | | |
| Myostatin | F | TCACGCTACCACGGAAACAA | 60 | 166 |
| | R | AGGAGTCTTGACGGGTCTGA | | |
| IGF1 | F | GGGGACTTTCGTGACTGAGC | 60 | 165 |
| | R | GGTAGGTCCGGGTCGTTTAC | | |
| Glyceral- | F | GTGATGGCATGGACTGTGGT | 55 | 163 |
| dehyde | R | GGAGCCAAAAGGGTCATCAT | | |
| 3-phosphate | | | | |
| dehydro- | | | | |
| genase | | | | |
| (GAPDH) | | | | |

2) Western Blotting

In order to perform Western blotting in cells, to each well from which the medium was removed was added a lysis buffer that contains 500 μL of 100 mM Tris-HCl, pH 7.4, 5 mM EDTA, 50 mM sodium pyrophosphate, 50 mM NaF, 100 mM orthovanadate, 1% Triton X-100, 1 mM phenylmethanesulfonyl fluoride, 2 μg/mL aprotinin, 1 μg/mL pepstatin A, and 1 μg/mL leupeptin, and the cells were lysed to obtain a lysate. Then, the lysate was centrifuged at 1,300×g and 4° C. for 20 minutes. Then, the middle layer was taken and proteins were quantified according to the Bradford method (Bio-Rad). 40 μg of the quantified proteins were electrophoresed on SDS polyacrylamide gel and then transferred to nitrocellulose membranes (Amersham, Buckinghamshire, UK). The membranes were repeatedly washed three times for 10 minutes each using a solution of Tris-buffered saline with Tween 20 (TBS-T), and then blocked using 10% skimmed milk for 60 minutes. Thereafter, the membranes were placed in primary antibodies diluted at a ratio of 1:1,000, gently shaken at 4° C., and incubated for 12 hours. Then, the membranes were washed with TBS-T. The membranes were again incubated with secondary antibodies diluted at a ratio of 1:2,000 for 60 minutes and washed. Here, as the primary antibodies, S6K1, phospho-p70S6K1 (p-p70S6K1), 4E-BP1, phospho-4E-BP1 (p-4E-BP1), and GAPDH (Cell Signaling Technology, Beverly, Mass., USA) were used. Finally, the proteins were visualized on an X-ray film using an ECL Western blot detection kit (RPN2106, Amersham, Arlington Heights, Ill., USA). Bands visualized on the X-ray film were scanned, and then quantified using the Quantity One analysis software (Bio-Rad).

2-2. Experiment Results

1) Identification of Changes in Expression of Protein Synthesis and Degradation-Related Molecules

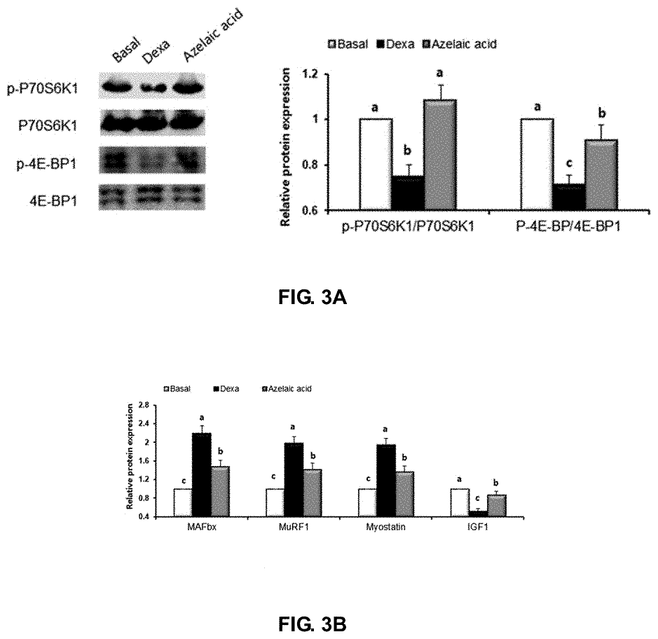

In this experiment, changes in expression of protein synthesis and degradation-related molecules, which are caused by azelaic acid, on mouse myoblasts were identified. In a case of dexamethasone-treated control cells (Dexa), amounts of p-p70S6K1 and p-4E-BP1 proteins associated with protein synthesis were significantly decreased as compared with normal cells (Basal) having no treatment with dexamethasone, whereas expression of the protein degradation genes MaFbx/Atrogin1, MuRF1, and myostatin was significantly increased and expression of the muscle-potentiating gene IGF1 was decreased. In a case of being treated with azelaic acid, the amounts of p-p70S6K1 and p-4E-BP1 proteins, and the expression of IGF1, which had decreased due to dexamethasone, were significantly increased again, and expression of MuRF1, MAFbx/Atrogin1, and myostatin was significantly decreased (FIGS. 3A-3B). Therefore, it is considered that in mouse myoblasts, azelaic acid increases p70S6K1 and 4E-BP1 protein phosphorylation and expression of IGF1 gene while inhibiting expression of myostatin gene, so that the azelaic acid might be ultimately involved in increasing muscle mass.

Hereinafter, preparation examples for medicines, foods, or cosmetics, which comprise the azelaic acid as an active ingredient, according to the present disclosure are described. However, such examples are not intended to limit the present disclosure but only to specifically illustrate the present disclosure. Medicine, food, or cosmetic compositions of Preparation Examples 1 to 3 were prepared in compliance with the following compositional ingredients and compositional proportions using the extract having a superior effect of preventing or treating a muscle disease, or improving muscle function.

### Preparation Example 1

### Preparation of Pharmaceutical Compositions

<1-1> Preparation of Powders

| | | |
|---|---|---|
| Azelaic acid | 20 | mg |
| Lactose hydrate | 100 | mg |
| Talc | 10 | mg |

The above ingredients were mixed, and an airtight bag was filled with the mixture to prepare powders.

<1-2> Preparation of Tablets

| | | |
|---|---|---|
| Azelaic acid | 10 | mg |
| Corn starch | 100 | mg |
| Lactose hydrate | 100 | mg |
| Magnesium stearate | 2 | mg |

The above ingredients were mixed, and then tableted according to a conventional tablet preparation method, to prepare tablets.

<1-3> Preparation of Capsules

| | | |
|---|---|---|
| Azelaic acid | 10 | mg |
| Microcrystalline cellulose | 3 | mg |
| Lactose hydrate | 14.8 | mg |
| Magnesium Stearate | 0.2 | mg |

The above ingredients were mixed, and then gelatin capsules were filled with the mixture according to a conventional capsule preparation method, to prepare capsules.

<1-4> Preparation of Injections

| | | |
|---|---|---|
| Azelaic acid | 10 | mg |
| Mannitol | 180 | mg |
| Sterile distilled water for injection | 2,974 | mg |
| Sodium monohydrogen phosphate | 26 | mg |

The above ingredients were mixed, and then the mixture was made into injections to have the above contents of ingredients per ampoule (2 ml) according to a conventional injection preparation method.

<1-5> Preparation of Liquids

| | | |
|---|---|---|
| Azelaic acid | 10 | mg |
| Isomerized sugar | 10 | mg |
| Mannitol | 5 | mg |

| | |
|---|---|
| Purified water | adequate amount |
| Lemon flavor | adequate amount |

According to a conventional preparation method, the above respective ingredients were added in purified water and dissolved therein. An adequate amount of the lemon flavor was added therein, and then purified water was added to adjust a total amount to 100 mL. Then, sterilization was performed and a brown bottle was filled with the resultant, to prepare liquids.

### Preparation Example 2

### Preparation of Health Foods

<2-1> Preparation of Health Supplement Food

| Aazelaic acid | 10 | mg |
|---|---|---|
| Vitamin mixture | adequate amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Mineral mixture | adequate amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 30 | mg |
| Calcium carbonate | 100 | mg |
| Magnesium chloride | 24.8 | mg |

For compositional proportions of the above-mentioned vitamin and mineral mixtures, ingredients that are relatively suitable for health foods were mixed in a preferred embodiment. However, blending proportions thereof may be changed in a predetermined manner for practicing the present disclosure. According to a conventional health food preparation method, the above ingredients may be mixed, and then granules may be prepared therefrom. The granules may be used for preparing health food compositions according to a conventional method.

<2-2> Preparation of Health Beverages

| | | |
|---|---|---|
| Azelaic acid | 10 | mg |
| Vitamin C | 15 | g |
| Vitamin E (powder) | 100 | g |
| Iron lactate | 19.75 | g |
| Zinc oxide | 3.5 | g |
| Nicotinic acid amide | 3.5 | g |
| Vitamin A | 0.2 | g |
| Vitamin B1 | 0.25 | g |
| Vitamin B2 | 0.3 | g |

| | |
|---|---|
| Purified water | adequate amount |

According to a conventional health beverage prepration method, the above ingredients were mixed and then the mixture was stirred and heated at 85° C. for about 1 hour. Then, the resulting solution was filtered and brought into a 2-L sterilized container. The container was sealed and sterilized, and refrigerated. Then, the solution was used for preparing a health beverage composition of the present disclosure.

For the above-mentioned compositional proportions, ingredients that are relatively suitable for favorite beverages were mixed in a preferred embodiment. However, blending proportions thereof may be changed in a predetermined manner for practicing the present disclosure, depending on regional or national preference such as demanding classes, demanding countries, and intended uses.

### Preparation Example 3

### Preparation of Cosmetic Compositions

Hereinafter, preparation examples of cosmetic compositions containing an extract of the present disclosure will be described. However, such examples are not intended to limit the present disclosure but only to specifically illustrate the present disclosure.

<3-1> Nourishing Lotion (Milk Lotion)

| | |
|---|---|
| Azelaic acid | 2.0% by weight |
| Squalane | 5.0% by weight |
| Beeswax | 4.0% by weight |
| Polysorbate 60 | 1.5% by weight |
| Sorbitan sesquioleate | 1.5% by weight |
| Liquid paraffin | 0.5% by weight |
| Caprylic or capric triglyceride | 5.0% by weight |
| Glycerine | 3.0% by weight |
| Butylene glycol | 3.0% by weight |
| Propylene glycol | 3.0% by weight |
| Carboxyvinyl polymer | 0.1% by weight |
| Triethanolamine | 0.2% by weight |
| Preservative, pigment, and flavoring agent | adequate amount |
| Purified water | to 100% by weight |

For the above-mentioned compositional proportions, ingredients that are relatively suitable for nourishing lotion were mixed in a preferred embodiment. However, blending proportions thereof may be changed in a predetermined manner for practicing the present disclosure, and preparation may be carried out according to a conventional preparation method in the cosmetic field.

<3-2> Softening Lotion (Skin Lotion)

| | | |
|---|---|---|
| Azelaic acid | 2.0% | by weight |
| Glycerin | 3.0% | by weight |
| Butylene glycol | 2.0% | by weight |
| Propylene glycol | 2.0% | by weight |
| Carboxyvinyl polymer | 0.1% | by weight |
| PEG 12 nonylphenyl ether | 0.2% | by weight |
| Polysorbate 80 | 0.4% | by weight |
| Ethanol | 10.0% | by weight |
| Triethanolamine | 0.1% | by weight |

| | |
|---|---|
| Preservative, pigment, and flavoring agent | adequate amount |
| Purified water | to 100% by weight |

For the above-mentioned compositional proportions, ingredients that are relatively suitable for softening lotion were mixed in a preferred embodiment. However, blending proportions thereof may be changed in a predetermined manner for practicing the present disclosure, and preparation may be carried out according to a conventional preparation method in the cosmetic field.

<3-3> Nourishing Cream

| | | |
|---|---|---|
| Azelaic acid | 2.0% | by weight |
| Polysorbate 60 | 1.5% | by weight |
| Sorbitan sesquioleate | 0.5% | by weight |
| PEG 60 hydrogenated castor oil | 2.0% | by weight |
| Liquid paraffin | 10% | by weight |
| Squalane | 5.0% | by weight |
| Caprylic or capric triglyceride | 5.0% | by weight |
| Glycerin | 5.0% | by weight |
| Butylene glycol | 3.0% | by weight |
| Propylene glycol | 3.0% | by weight |
| Triethanolamine | 0.2% | by weight |

| | |
|---|---|
| Preservative | adequate amount |
| Pigment | adequate amount |
| Flavoring agent | adequate amount |
| Purified water | to 100% by weight |

For the above-mentioned compositional proportions, ingredients that are relatively suitable for nourishing cream were mixed in a preferred embodiment. However, blending proportions thereof may be changed in a predetermined manner for practicing the present disclosure, and preparation may be carried out according to a conventional preparation method in the cosmetic field.

<3-4> Massage Cream

| | | |
|---|---|---|
| Azelaic acid | 1.0% | by weight |
| Beeswax | 10.0% | by weight |
| Polysorbate 60 | 1.5% | by weight |
| PEG 60 hydrogenated castor oil | 2.0% | by weight |
| Sorbitan sesquioleate | 0.8% | by weight |
| Lquid paraffin | 40.0% | by weight |
| Squalane | 5.0% | by weight |
| Caprylic or capric triglyceride | 4.0% | by weight |
| Glycerin | 5.0% | by weight |
| Butylene glycol | 3.0% | by weight |
| Propylene glycol | 3.0% | by weight |
| Triethanolamine | 0.2% | by weight |

| | |
|---|---|
| Preservative, pigment, and flavoring agent | adequate amount |
| Purified water | to 100% by weight |

For the above-mentioned compositional proportions, ingredients that are relatively suitable for massage cream were mixed in a preferred embodiment. However, blending proportions thereof may be changed in a predetermined manner for practicing the present disclosure, and preparation may be carried out according to a conventional preparation method in the cosmetic field.

<3-5> Pack

| | | |
|---|---|---|
| Azelaic acid | 1.0% | by weight |
| Polyvinyl alcohol | 13.0% | by weight |
| Sodium carboxymethylcellulose | 0.2% | by weight |
| Glycerin | 5.0% | by weight |
| Allantoin | 0.1% | by weight |
| Ethanol | 6.0% | by weight |
| PEG 12 nonylphenyl ether | 0.3% | by weight |
| Polysorbate 60 | 0.3% | by weight |

| | |
|---|---|
| Preservative, pigment, and flavoring agent | adequate amount |
| Purified water | to 100% by weight |

For the above-mentioned compositional proportions, ingredients that are relatively suitable for pack were mixed in a preferred embodiment. However, blending proportions thereof may be changed in a predetermined manner for practicing the present disclosure, and preparation may be carried out according to a conventional preparation method in the cosmetic field.

<3-6> Gel

| | | |
|---|---|---|
| Azelaic acid | 0.5% | by weight |
| Eethylenediamine sodium acetate | 0.05% | by weight |
| Glycerin | 5.0% | by weight |
| Carboxyvinyl polymer | 0.3% | by weight |
| Ethanol | 5.0% | by weight |
| PEG 60 hydrogenated castor oil | 0.5% | by weight |
| Triethanolamine | 0.3% | by weight |

| | |
|---|---|
| Preservative, pigment, and flavoring agent | adequate amount |
| Purified water | to 100% by weight |

For the above-mentioned compositional proportions, ingredients that are relatively suitable for gel were mixed in a preferred embodiment. However, blending proportions thereof may be changed in a predetermined manner for practicing the present disclosure, and preparation may be carried out according to a conventional preparation method in the cosmetic field.

For the above-mentioned compositional proportions, ingredients that are relatively suitable for cosmetic compositions were mixed in a preferred embodiment. However, the present disclosure may be applied to cosmetics for various uses including other color cosmetics, and may be used for preparing a medicament which can be applied thinly on a human body, that is, an ointment, depending on efficacy thereof. Blending proportions thereof may be changed in a predetermined manner for practicing the present disclosure, depending on regional or national preference such as demanding classes, demanding countries, and intended uses.

The foregoing description of the present disclosure is provided for illustration. It will be understood by those skilled in the art that various changes and modifications can be easily made without departing from the technical spirit or essential features of the present disclosure. Therefore, it is to be understood that the above-described examples are illustrative in all aspects and not restrictive.

1. A method for preventing or treating a muscle disease, the method comprising administering a pharmaceutical composition including azelaic acid or a pharmaceutically acceptable salt thereof as an active ingredient.

2. The method according to claim 1, wherein the azelaic acid or a pharmaceutically acceptable salt thereof increases expression of p-4E-BP1 and p-p70S6K1 proteins.

3. The method according to claim 1, wherein the azelaic acid or a pharmaceutically acceptable salt thereof decreases expression of muscle RING-finger protein (MuRF1), muscle atrophy F-box (MaFbx), or myostatin.

4. The method according to claim 1, wherein the muscle disease is a muscle disease caused by muscle function decline, muscle decrease, muscle atrophy, muscle wasting, or muscle degeneration.

5. The method according to claim 1, wherein the muscle disease is any one or more selected from the group consisting of atony, muscular atrophy, muscular dystrophy, myasthenia, cachexia, rigid spine syndrome, amyotrophic lateral sclerosis (Lou Gehrig's disease), Charco-Marie-Tooth disease, and sarcopenia.

6. The method for promoting muscle differentiation, regenerating muscles, or enhancing muscles, the method comprising administering a composition including azelaic acid or a pharmaceutically acceptable salt thereof as an active ingredient, or allowing it to be taken.

7. The method according to claim 6, wherein the composition is pharmaceutical composition or health functional food composition.

8. A method for increasing muscle mass or promoting muscle production, the method comprising administering a composition including azelaic acid or a pharmaceutically acceptable salt thereof as an active ingredient, or allowing it to be taken.

9. The method according to claim 8, wherein the composition is pharmaceutical composition or health functional food composition.


SEQUENCE LISTING (10 sequences)

SEQ ID NO: 1 - DNA, 20 bp, Artificial
  MaFbx_F primer
gtccagagagtcggcaagtc

SEQ ID NO: 2 - DNA, 20 bp, Artificial
  MaFbx_R primer
gtcggtgatcgtgagacctt

SEQ ID NO: 3 - DNA, 20 bp, Artificial
  MuRF1_F primer
acatctactgtctcacgtgt

SEQ ID NO: 4 - DNA, 20 bp, Artificial
  MuRF1_R primer
tgtccttggaagatgctttg

SEQ ID NO: 5 - DNA, 20 bp, Artificial
  Myostatin_F primer
tcacgctaccacggaaacaa

SEQ ID NO: 6 - DNA, 20 bp, Artificial
  Myostatin_R primer
aggagtcttgacgggtctga

SEQ ID NO: 7 - DNA, 20 bp, Artificial
  IGF1_F primer
ggggactttcgtgactgagc

SEQ ID NO: 8 - DNA, 20 bp, Artificial
  IGF1__R primer
ggtaggtccgggtcgtttac

SEQ ID NO: 9 - DNA, 20 bp, Artificial
  GAPDH_F primer
gtgatggcatggactgtggt

SEQ ID NO: 10 - DNA, 20 bp, Artificial
  GAPDH_R primer
ggagccaaaagggtcatcat